(19) United States
(12) Patent Application Publication
(10) Pub. No.: US 2024/0337650 A1
(43) Pub. Date: Oct. 10, 2024

(54) A Paper-Based Sensor

(71) Applicant: AGENCY FOR SCIENCE, TECHNOLOGY AND RESEARCH, Singapore (SG)

(72) Inventors: Xinting Zheng, Singapore (SG); Laura Sutarlie, Singapore (SG); Yong Yu, Singapore (SG); Xiaodi Su, Singapore (SG)

(21) Appl. No.: 18/574,424

(22) PCT Filed: Jun. 27, 2022
(86) PCT No.: PCT/SG2022/050439

§ 371 Date: Dec. 27, 2023

(30) Foreign Application Priority Data
Jun. 28, 2021 (SG) 10202107044R

(51) Int. Cl. G01N 33/52; G01N 33/53; G01N 33/84

(52) U.S. Cl. CPC G01N 33/523; G01N 33/5308; G01N 33/84; G01N 2800/202

(57) ABSTRACT

There is provided herein a paper-based sensor for simultaneously determining a plurality of biomarkers present in a biological sample comprising a plurality of detection zones in fluid communication with a sampling zone, wherein said plurality of detection zones comprises sensing material specific to each of said plurality of biomarkers. There is also provided herein a method of manufacturing the paper-based sensor, a use of a paper-based sensor for wound diagnosis, a kit comprising the paper-based sensor and a method of diagnosing wound health.

### CROSS-REFERENCE TO RELATED APPLICATIONS

This application claims priority to Singapore provisional application Ser. No. 10/202,107,044R filed on 28 Jun. 2021 with the Intellectual Property Office of Singapore, the contents of which are incorporated by reference.

### TECHNICAL FIELD

The present invention relates generally to a paper-based sensor for simultaneously determining a plurality of biomarkers present in a biological sample and a method of manufacturing the sensor as described herein. The present invention also relates to a use of the paper-based sensor as described herein for wound monitoring and wound diagnosis. The present invention also relates to a kit comprising the paper-based sensor as described herein, a device for capturing images and a software for image analysis.

### BACKGROUND ART

In current wound care practices, the monitoring of wound healing is typically performed visually by clinicians. Wound infections are most commonly diagnosed via “swabbing” or wound biopsy, followed by a culture-based assessment which usually requires 24 hours of incubation. A few limitations of current wound monitoring measures include a lack of holistic profiling and quantitative characterization of wound for inflammation, infections and vascularization during wound healing; frequent manual removal of wound covers which elevates the risks of infection and added trauma; and long turnover times of hours and days due to culturing bacteria to assess the infection level of a wound.

In general, the wound healing process consists of three phases: haemostasis and inflammation, proliferation, and tissue remodelling. As such, the complex nature of the wound healing process requires monitoring of several diagnostic markers. A list of known biomarkers and its clinical significance are laid out in Table 1. below.

TABLE 1
Summary of wound biomarkers

| Biomarker | Clinical significance | Normal range | Unhealthy range |
|---|---|---|---|
| Temperature | Persistent increased temperature | 31° C. to 35° C. | 1° C. increase relative |
| | (above 1° C.) adjacent to wound is a | | to normal baseline |
| | predictive sign of infection and | | level |
| | delayed healing. | | |
| Trimethylamine | Trimethylamine is a metabolite | Not detected | 30 ppm to 300 ppm |
| (TMA) | produce by most bacteria in wounds | | |
| pH | Increase in wound pH is an indicator | pH 6 to pH 8 with | pH 6 to pH 9.5 with a |
| | of local wound infection | a mean of pH 6.7 | mean of pH 8.1 |
| Moisture | Wound moisture levels are known | 0% to 100% | |
| | to be critical to healing. Too much | Relative | |
| | moisture can result in maceration | Humidity; | |
| | while too little moisture can lead to | varies by wound | |
| | wound drying out. | dressing | |
| Uric acid | S. aureus and P. aeruginosa rapidly | 221 μM to 751 | 50% to 100% relative |
| | metabolize uric acid via uricase | μM with 347 μM | decrease with respect |
| | synthesis, therefore a rapid | median value | to baseline level |
| | decrease in uric acid concentration | | indicates bacterial |
| | within wound exudate may be a | | infection. |
| | generic indicator for bacterial | | Significant increase |
| | colonization | | with respect to |
| | | | baseline level may be |
| | | | linked to |
| | | | inflammation. |

Current sensors used to determine a few of these biomarkers tend to be electrochemical sensors or a combination of electrochemical sensors with optical sensors deployed for monitoring of multiple biomarkers. The use of electrochemical sensing principles has certain drawbacks, for example, requiring many components including printed circuit boards, blue-tooth configuration, reliance on battery etc., resulting in a sensor made up of many parts which can be bulky and inconvenient to use if a power source is absent.

Therefore, there is a need to provide a holistic sensor that can determine and/or analyse wound biomarkers, such as those listed in Table 1., quickly and without causing additional trauma to the wound.

### SUMMARY

In one aspect, the present disclosure relates to paper-based sensor for simultaneously determining a plurality of biomarkers present in a biological sample, said paper-based sensor comprises:
a) a sample zone for receiving said biological sample containing said plurality of biomarkers; and
b) a plurality of detection zones in fluid communication with said sample zone, each of said plurality of detection zones comprising sensing material specific to said plurality of biomarker,
wherein said plurality of biomarkers are selected from the group consisting of temperature, trimethylamine (TMA), pH, moisture and uric acid.

Advantageously, the paper-based sensor may provide a quantitative characterization of multiple wound biomarkers simultaneously, such as temperature, trimethylamine, pH, moisture and uric acid, to monitor wound infection and inflammation.

Further advantageously, the paper-based sensor may be manufactured into a flexible wound sensor patch which can be integrated in combination with other wound dressings for in-situ analysis without the need to remove the wound dressing. As such, the risk of wound infection and additional trauma to the wound may be significantly reduced.

Further advantageously, the paper-based sensor may provide a quicker assessment of the wound health within minutes and may also provide a real-time assessment of wound healing.

Further advantageously, as the paper-based sensor is not an electrochemical sensor, the paper-based sensor may not require a power source to work, may be light-weight and can be easily integrated into various kinds of wound dressing for in situ detection.

In another aspect, the present disclosure relates to a method of manufacturing the paper-based sensor as described herein, comprising the steps of:
a) providing a pattern having a first area and a second area on a base substrate, wherein the first area defines a sample zone and a plurality of detection zones in fluid communication with the sample zone and wherein the second area is the remaining portion of the base substrate that is not covered by the first area;
b) printing a hydrophobic material or ink onto the second area of the pattern of step (a);
c) heating the hydrophobic material or ink in the second area to demarcate the sample zone and the plurality of detection zones in the first area; and
d) treating each of the plurality of detection zones with a sensing material that is specific to a biomarker selected from the group consisting of temperature, trimethylamine (TMA), pH, moisture and uric acid.

Advantageously, the paper-based sensor as prepared by the method described herein may be stored at room temperature for at least two weeks without significant decay in the enzyme activity.

In another aspect, the present disclosure relates to a use of the paper-based sensor as described herein for wound monitoring.

In another aspect, the present disclosure relates to a kit comprising the paper-based sensor as described herein, a device for capturing images and a software for image analysis.

In another aspect, the present disclosure relates to a method of diagnosing wound health, comprising the step of applying the paper-based sensor as described herein onto a wound directly or in combination with other wound dressings.

### Definitions

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DESCRIPTION OF OPTIONAL EMBODIMENTS

Exemplary, non-limiting embodiments of a paper-based sensor for simultaneously determining a plurality of biomarkers present in a biological sample will now be disclosed.

The paper-based sensor comprises:
a) a sample zone for receiving the biological sample containing the plurality of biomarkers; and
b) a plurality of detection zones in fluid communication with the sample zone, each of the plurality of detection zones comprising sensing material specific to the plurality of biomarker, wherein the plurality of biomarkers are selected from the group consisting of temperature, trimethylamine (TMA), pH, moisture and uric acid.

The paper-based sensor may comprise a base selected from a cellulose base, a nitrocellulose base or a glass microfiber base. The cellulose base may be a cellulose paper base with particle retention of about 5 μm to about 7 μm, about 5 μm to about 6 μm, or about 6 μm to about 7 μm.

The base may have a thickness of about 300 μm to about 450 μm, about 350 μm to about 450 μm, about 400 μm to about 450 μm, about 350 μm to about 450 μm, about 400 μm to about 450 μm, or about 380 μm to about 400 μm.

Each of the detection zone may independently have a diameter of about 2 mm to about 20 mm, about 5 mm to about 20 mm, about 10 mm to about 20 mm, about 15 mm to about 20 mm, about 2 mm to about 15 mm, about 2 mm to about 10 mm, about 2 mm to about 5 mm or about 4 mm to about 6 mm.

Where the detection zone is in fluid communication with the sample zone, the detection zones may be connected to the sample zone via respective channels. The channels may have a width of about 1 mm to about 10 mm, about 2 mm to about 10 mm, about 4 mm to about 10 mm, about 6 mm to about 10 mm, about 2 mm to about 8 mm, about 2 mm to about 6 mm or about 2 mm to about 4 mm. The channel widths may be predetermined by computer-aided design (CAD) and printed as such. The channel width may be at least 1 mm to allows viscous wound exudate to flow through.

The sample zone may have a diameter of about 2 mm to about 20 mm, about 5 mm to about 20 mm, about 10 mm to about 20 mm, about 15 mm to about 20 mm, about 2 mm to about 15 mm, about 2 mm to about 10 mm, about 2 mm to about 5 mm or about 4 mm to about 6 mm. The sample zone may receive the biological sample from the front (or top) or back (or bottom) of the paper-based sensor.

Each of the detection zones may extend radially outwards from the sample zone. The sample zone may then be viewed as being placed in the central position of the sensor with channels in-between and connecting the sample zone to each of the detection zones. When the biological fluid is deposited onto the sample zone, the biological fluid may travel along the channels to the detection zones at a consistent rate across the various channels.

The paper-based sensor may further comprise a hydrophobic region surrounding the sample zone and the detection zones, whereby the hydrophobic region comprises a hydrophobic material. The hydrophobic material may be a wax to prevent cross-movement or cross-contamination between the detection zones.

The hydrophobic material may be heated at a suitable temperature (such as about 90° C.) for a duration of about 5 minutes to about 30 minutes, about 10 minutes to about 30 minutes, about 15 minutes to about 30 minutes, about 20 minutes to about 30 minutes, about 25 minutes to about 30 minutes, about 5 minutes to about 25 minutes, about 5 minutes to about 20 minutes, about 5 minutes to about 15 minutes or about 5 minutes to about 10 minutes. The heating duration may control the width of the channels formed. The longer the duration of heating the hydrophobic material, the more the hydrophobic material will seep into the paper base, causing the narrowing of the channels.

The paper-based sensor may be integrated between a top transparent silicone layer, a bottom transparent silicone layer with a central circular opening, and a blood filtration membrane located between the central circular opening of bottom transparent silicone layer and the paper-based sensor. This integrated paper-based sensor may be termed as a wound sensor patch.

The top silicone layer may be medical grade silicone MDX4-4210 or 3M™ Tegardem™ Transparent Film. The bottom silicone layer may be medical grade silicone MG7-9800, 3M™ Tegardem™ Transparent Film or Mepitel Silicone Wound Contact Layer. The bottom silicone layer may further comprise an adhesive layer.

The blood filtration membrane may be Whatman MF1 or Whatman LF1 blood filtration membrane.

Advantageously, the silicone layers add a longer shelf-life to the paper-based sensor and the blood filtration membrane confers the ability to remove the red colour from blood-stained wound exudates.

When the biomarker is temperature, the sensing material of the temperature biomarker may be a mixture of two or more cholesteric liquid crystals (CLCs). The CLCs may be cholesterol derivatives selected from the group comprising of cholesteryl oleyl carbonate (COC), cholesteryl benzoate (CB), cholesteryl nonanoate (CN), cholesteryl pelargonate (CP) and cholesteryl chloride (CC). As an example, the sensing material of the temperature biomarker may comprise 36% COC, 10% CB and 54% CN.

The mixture of CLCs may be dropped and spread onto the cellulose paper detection zone. The detection zone may be treated with black ink and dried at room temperature for about 5 minutes to about 20 minutes to evaporate solvent from the ink and prevent contamination to the CLCs. The CLC colour may be best viewed against a non-reflecting background.

When the biomarker is TMA, the sensing material of the TMA biomarker may comprise a solution of solvatochromic dyes selected from the group consisting of Reichardt's dye, Nile Red, 3-hydroxychromones, merocyanine and azomerocyanine betaine. The solvatochromic dye may be Reichardt's dye dissolved in an alcohol in a concentration of about 1 mg/ml to about 10 mg/mL, about 2 mg/mL to about 10 mg/mL, about 4 mg/mL to about 10 mg/mL, about 6 mg/mL to about 10 mg/mL, about 8 mg/mL to about 10 mg/mL, about 2 mg/mL to about 8 mg/mL, about 2 mg/mL to about 6 mg/mL or about 4 mg/mL to about 6 mg/mL.

The alcohol may be selected from the group consisting of methanol, ethanol, propanol, iso-propanol, or any combination thereof. The alcohol may be suitable to dissolve the solvatochromic dye.

The sensing material of the TMA sensor comprising the solution of Reichardt's dye in alcohol may be drop-casted to the TMA detection zone treated with about 1% to about 10% (such as 1%) perfluorooctyl-trimethoxy silane.

The sensing material of the TMA sensor may be dried at a temperature range of about 25° C. to about 50° C., about 30° C. to about 50° C., about 35° C. to about 50° C., about 40° C. to about 50° C., about 45° C. to about 50° C., about 25° C. to about 45° C., about 25° C. to about 40° C., about 25° C. to about 35° C. or about 25° C. to about 30° C. The temperature may be room temperature to about 50° C. The sensing material of the TMA sensor may be dried for a duration of about 5 minutes to about 20 minutes, about 10 minutes to about 20 minutes, about 15 minutes to about 20 minutes, about 5 minutes to about 15 minutes or about 5 minutes to about 10 minutes.

The TMA sensor may be calibrated by capturing digital images of the colour change and processing the image using an image processing software to obtain the Red-Blue-Green (RGB) and luminance values. The luminance values may be used to correlate the colour change with the measured concentration of measured TMA. The digital images may be captured using a mobile device, a mobile phone camera or a digital camera and the image processing software may be ImageJ or GIMP.

When the biomarker is pH, the sensing material of the pH biomarker may comprise phenol red, neutral red or bromothymol blue. Apart from producing evident colour change to changes in pH, the biocompatibility and toxicity of the pH sensor must also be considered, as such the pH sensor may be phenol red. The pH sensor may comprise phenol red powder dissolved in water (forming aqueous phenol red) at a concentration of about 0.02% to about 0.10%, about 0.04% to about 0.10%, about 0.06% to about 0.10. %, about 0.08% to about 0.10%, about 0.02% to about 0.08%, about 0.02% to about 0.06% or about 0.02% to about 0.04%.

The aqueous phenol red, neutral red or bromothymol blue may be drop-casted onto the pH detection zone and dried at room temperature to produce one layer of pH sensor. The pH sensor may comprise one or more layers.

The sensing material of the pH sensor may be dried for a duration of about 5 minutes to about 20 minutes, about 10 minutes to about 20 minutes, about 15 minutes to about 20 minutes, about 5 minutes to about 15 minutes or about 5 minutes to about 10 minutes.

The pH sensor may be calibrated by capturing digital images of the colour change and processing the image using an image processing software to obtain the Red-Blue-Green (RGB) values. The ratio of B/G values may be used to correlate the colour change with the measured pH. The digital images may be captured using a mobile device, a mobile phone camera or a digital camera and the image processing software may be ImageJ or GIMP.

When the biomarker is moisture, the sensing material of the moisture biomarker may comprise a transition metal salt dissolved in a mixture of polyhydroxyethylmethacrylate (pHEMA)/alcohol solution at a concentration of about 10 mg/mL to about 200 mg/mL, about 20 mg/mL to about 200 mg/mL, about 100 mg/mL to about 200 mg/mL, about 150 mg/mL to about 200 mg/mL, about 10 mg/mL to about 150 mg/mL, about 10 mg/mL to about 100 mg/mL, about 10 mg/mL to about 30 mg/mL, about 20 mg/mL to about 30 mg/mL and about 10 mg/mL to about 20 mg/mL. The alcohol may be methanol, ethanol, propanol or isopropyl alcohol (IPA). The concentration of the pHEMA/alcohol solution may be 20 mg/mL (2 weight %). The transition metal concentration salt may be selected from the Group 8, 9, 10 or 11 of the Periodic Table of Elements and may be a halide salt. The transition metal salt may be cobalt chloride (CoCl₂), cobalt bromide (CoBr₂) or copper chloride (CuCl₂). The transition metal salt may be cobalt chloride.

About 10 μL/cm² to about 30 μL/cm² of the solution of transition metal salt dissolved in pHEMA/alcohol solution may be drop-casted onto the moisture detection zone and dried in an oven at a temperature for a duration at an appropriate fan speed (such as 100% fan) to produce one layer of moisture sensor. The moisture sensor may comprise one or more layers.

The oven temperature may be about 20° C. to about 100° C., about 25° C. to about 100° C., about 50° C. to about 100° C., about 75° C. to about 100, about 20° C. to about 75° C. and about 20° C. to about 50° C. The duration may be about 5 minutes to about 20 minutes, about 10 minutes to about 20 minutes, about 15 minutes to about 20 minutes, about 5 minutes to about 15 minutes or about 5 minutes to about 10 minutes.

Advantageously, the pHEMA provides a three-dimensional network for the transition metal salt (such as CoCl₂) and its hygroscopic property speeds up water absorption to render the moisture sensor to be efficient in providing a colour change response to the amount of moisture present in the wound.

The moisture sensor may be calibrated by capturing digital images of the colour change and processing the image using an image processing software to obtain the Red-Blue-Green (RGB) values. The ratio of R/B values may be used to correlate the colour change with the measured moisture percentage. The digital images may be captured using a mobile device, a mobile phone camera or a digital camera and the image processing software may be ImageJ or GIMP.

When the biomarker is uric acid, the sensing material of the uric acid biomarker may be uricase enzyme in an enzymatic substrate in biopolymer matrix, wherein the uricase enzyme may be stabilised by an immunoassay stabiliser. The uric acid sensor may also comprise of horseradish peroxidase (HRP) and wherein the HRP may be stabilised by an immunoassay stabiliser.

The biopolymer matrix may be selected from the group comprising of collagen or chitosan. The molecular weight of the biopolymer matrix may be in the range of about 20 000 Daltons to about 200 000 Daltons, about 50 000 Daltons to about 200 000 Daltons, about 100 000 Daltons to about 200 000 Daltons, about 150 000 Daltons to about 200 000 Daltons, about 20 000 Daltons to about 150 000 Daltons, about 20 000 Daltons to about 100 000 Daltons or about 20 000 Daltons to about 50 000 Daltons. The biopolymer matrix may be low molecular weight chitosan. The biopolymer matrix may be prepared at a concentration of about 1 weight % to about 10 weight %, about 2 weight % to about 10 weight %, about 5 weight % to about 10 weight %, about 8 weight % to about 10 weight %, about 1 weight % to about 8 weight %, about 1 weight % to about 5 weight % and about 1 weight % to about 2 weight %, based on the weight of chitosan. The chitosan may be dissolved in 1 weight % acetic acid, based on the weight of acetic acid.

The low molecular chitosan may be prepared at a pH of about pH 5.5 to about pH 7.5, about pH 5.5 to about pH 6.5 and about pH 6.5 to about pH 7.5.

The enzymatic substrate may be selected from the group consisting of 3,3′,5,5′-tetramethylbenzidine (TMB), 4-aminoantipyrine (AAP), o-phenylenediamine (OPD), o-dianisidine, 2,2′-Azino-Bis-3-Ethylbenzothiazoline-6-Sulfonic Acid (ABTS) and any combinations thereof. The enzymatic substrate may be AAP.

The enzymatic substrate may be prepared in sodium 3,5-dichloro-2-hydroxybenzenesulfonate (DHBS) at a molar concentration of about 10 mM to about 100 mM, about 20 mM to about 100 mM, about 40 mM to about 100 mM, about 60 mM to about 100 mM, about 80 mM to about 100 mM, about 10 mM to about 80 mM, about 10 mM to about 60 mM, about 10 mM to about 40 mM or about 10 mM to about 20 mM.

The AAP may be prepared in DHBS at a molar concentration of about 2 mM to about 20 mM, about 4 mM to about 20 mM, about 8 mM to about 20 mM, about 12 mM to about 20 mM, about 16 mM to about 20 mM, about 2 mM to about 16 mM, about 2 mM to about 12 mM, about 2 mM to about 8 mM or about 2 mM to about 4 mM.

The HRP may be prepared at a concentration of about 0.1 mg/mL to about 1 mg/mL, about 0.2 mg/mL to about 1 mg/mL, about 0.3 mg/mL to about 1 mg/mL, about 0.6 mg/mL to about 1 mg/mL, about 0.9 to about 1 mg/mL, about 0.1 mg/mL to about 0.9 mg/mL, about 0.1 mg/mL to about 0.6 mg/mL, about 0.1 mg/mL to about 0.3 mg/mL or about 0.1 mg/mL to about 0.2 mg/mL. The HRP may be stabilised in an immunoassay solution. The immunoassay solution may be StabilCoat® Immunoassay Stabilizer.

The uricase enzyme may be prepared at a concentration of about 10 mg/mL to about 100 mg/mL, about 20 mg/mL to about 100 mg/mL, about 40 mg/mL to about 100 mg/mL, about 60 mg/mL to about 100 mg/mL, about 80 mg/mL to about 100 mg/mL, about 10 mg/mL to about 80 mg/mL, about 10 mg/mL to about 60 mg/mL, about 10 mg/mL to about 40 mg/mL or about 10 mg/mL to about 20 mg/mL. The uricase enzyme may be stabilised in an immunoassay solution. The immunoassay solution may be StabilCoat® Immunoassay Stabilizer.

The uric acid sensing material may be 40 mg/mL uricase enzyme in StabilCoat® Immunoassay Stabilizer solution in 16 mM AAP dissolved in 8 mM DHBS in 1 weight % chitosan matrix at pH 6.5 with 0.15 mg/mL HRP in StabilCoat® Immunoassay Stabilizer solution.

Advantageously, the uric acid sensing material with 1 weight % chitosan at pH 6.5 shows good colour retention for up to 40 minutes.

Further advantageously, the enzymatic activity of uricase is preserved for at least two weeks at room temperature when stabilised with StabilCoat® Immunoassay Stabilizer solution.

Further advantageously, a good colour gradient is obtained when the uric acid sensing material is drop-casted onto the uric acid detection zone with particle retention of 6 μm and thickness of 390 μm.

Further advantageously, the uric acid sensing material does not give a false positive when prepared with AAP in DHBS.

The uric acid sensor may be calibrated by capturing digital images of the colour change and processing the image using an image processing software to obtain the intensity values. The ratio of R/B values may be used to correlate the colour change with the measured moisture percentage. The digital images may be captured using a mobile device, a mobile phone camera or a digital camera and the image processing software may be ImageJ or GIMP.

Exemplary, non-limiting embodiments of a method of manufacturing the paper-based sensor will now be disclosed.

The method may comprise the steps of:
a) providing a pattern having a first area and a second area on a base substrate, wherein the first area defines a sample zone and a plurality of detection zones in fluid communication with the sample zone and wherein the second area is the remaining portion of the base substrate that is not covered by the first area;
b) printing a hydrophobic material or ink onto the second area of the pattern of step (a);
c) heating the hydrophobic material or ink in the second area to demarcate the sample zone and the plurality of detection zones in the first area; and
d) treating each of the plurality of detection zones with a sensing material that is specific to a biomarker selected from the group consisting of temperature, trimethylamine (TMA), pH, moisture and uric acid.

The pattern in the providing step (a) may be drawn onto the base substrate using computer aided design (CAD) software. Advantageously, using CAD software to draw the pattern ensure that the dimensions of the channels, detections zones and sample zone are accurate.

The method may further comprise a step of preparing the sensing materials for the biomarkers before drawing step (a).

The treating step (d) may further comprise treating the detections zones sequentially, beginning with the sensing materials for TMA, followed by temperature, followed by pH, followed by moisture and followed by uric acid.

The treating step for the sensing material for the temperature biomarker may comprise the steps of:
i) melting a cholesteric liquid crystal mixture at a temperature of about 80° C. to about 120° C. (such as 100° C.) for about 30 minutes to about 2 hours (such as 1 hour);
ii) depositing a black ink on the temperature detection zone and drying for about 5 minutes to about 20 minutes (such as 15 minutes) at a temperature of about 25° C. to about 50° C. (such as room temperature); and
iii) dropping and spreading an amount of the melted CLC of step (a) onto the temperature detection zone of step (b).

The treating step for the sensing material of the trimethylamine biomarker may comprise the steps of:
i) dissolving about 1 mg/mL to about 10 mg/mL (such as 5 mg/mL) of a solvatochromic dye selected from the group consisting of Reichardt's dye, Nile Red and 3-hydroxychromones (such as Reichardt's dye) in alcohol (such as ethanol);
ii) treating the trimethylamine detection zone with about 1% to about 10% (such as 1%) perfluorooctyl-trimethoxy silane;
iii) drop casting about 10 μL/cm² to about 30 μL/cm² (such as 20 μL/cm²) of solution prepared in step (i) into the treated trimethylamine detection zone of step (ii); and
iv) drying the trimethylamine detection zone for about 5 minutes to about 20 minutes (such as 10 minutes) at a temperature of about 25° C. to about 50° C. (such as 40° C.) after step (iii).

The treating step for the sensing material of pH biomarker may comprise the steps of: i) dissolving about 0.02% to about 0.10% (such as 0.04%) of phenol red, neutral red or bromothymol blue (such as phenol red) in water to form a solution;
ii) drop casting about 10 μL/cm² to about 30 μL/cm² (such as 20 μL/cm²) of the solution from step (i) into the pH detection zone;
iii) air drying the pH detection zone in step (ii) for about 5 minutes to about 20 minutes (such as 5 minutes) at a temperature of about 25° C. to about 50° C. (such as room temperature) to create one layer; and
iv) repeating steps (ii) and (iii) to produce up to 3 layers.

The treating step for the sensing material for the moisture biomarker may comprise the steps of:
i) dissolving about 10 mg/mL to about 200 mg/mL (such as 100 mg/mL) of a transition metal salt (such as cobalt chloride (CoCl₂), cobalt bromide (CoBr₂) or copper chloride (CuCl₂)) in about 1 wt % to about 10 wt % in 2 wt % (20 mg/mL) polyhydroxyethylmethacrylate (pHEMA)/ethanol solution;
ii) drop casting about 10 μL/cm² to about 30 μL/cm² (such as 10 μL/cm²) of the solution prepared in step (i) into the moisture detection zone;
iii) drying the moisture detection zone in step (iii) in an oven at about 20° C. to about 30° C. (such as 25° C.) for about 5 minutes to about 20 minutes (such as 5 minutes) to create one layer; and
iv) repeating steps (ii) and (iii) to produce up to 3 layers.

The treating step for the sensing material for the uric acid biomarker may comprise the steps of:
i) adding about 10 μL/cm² to about 30 μL/cm² (such as 10 μL/cm²) of about 1 wt % to about 100 wt % (such as 1 wt %) of a biopolymer matrix (such as chitosan) with pH tuned in the range of about pH 5.5 to about pH 7.5 (such as pH 6.5) to the uric acid detection zone and drying for about 5 minutes to about 20 minutes (such as 5 minutes) at a temperature of about 25° C. to about 30° C. (such as room temperature);
ii) adding about 10 μL/cm² to about 30 μL/cm² (such as 10 L/cm²) of about 10 mM to about 100 mM (such as 16 mM) of 3,3′,5,5′-tetramethylbenzidine (TMB), 4-aminoantipyrine (AAP), o-phenylenediamine (OPD), o-dianisidine or 2,2′-Azino-Bis-3-Ethylbenzothiazoline-6-Sulfonic Acid (ABTS) in about 2 mM to about 20 mM (such as 8 mM) of 3,5-dichloro-2-hydroybenzenesulfonate (DHBS) to the uric acid detection zone in step (i) and drying for about 5 minutes to about 20 minutes (such as 5 minutes) at a temperature of about 25° C. to about 30° C. (such as room temperature);
iii) adding about 10 μL/cm² to about 30 μL/cm² (such as 10 μL/cm²) of about 0.1 mg/mL to about 1 mg/mL (such as 0.15 mg/mL) of horseradish peroxidase (HRP) in stabilizer solution to the uric acid detection zone in step (ii) and drying for about 5 minutes to about 20 minutes (such as 5 minutes) at a temperature of about 25° C. to about 30° C. (such as room temperature);
iv) adding about 10 μL/cm² to about 30 μL/cm² (such as 10 μL/cm²) of about 10 mg/mL to about 100 mg/mL (such as 40 mg/mL) of uricase in stabilizer solution to the uric acid detection zone in step (iii) and drying for about 5 minutes to about 20 minutes (such as 5 minutes) at a temperature of about 25° C. to about 30° C. (such as room temperature); and
v) re-adding of about 10 L/cm² to about 30 μL/cm² (such as 10 μL/cm²) of the AAP solution in step (ii) to the uric acid detection zone in step (iv) and drying for about 5 minutes to about 20 minutes (such as 5 minutes) at a temperature of about 25° C. to about 30° C. (such as room temperature).

The paper-based sensor may be formed into a wound sensor patch. Therefore, there is provided a method of forming a wound sensor patch, comprising the steps of:
i) fabricating a first and second silicone layer from medical grade silicone (such as MDX4-42210 or MG7-9800) by mixing a base elastomer component and a curing component in a ratio of about 30:1 to about 0.3:1 (such as 10:1 to 0.9:1);
ii) curing the first and second silicone layers of step (i) at a temperature in the range of about 25° C. to about 120° C. (such as 60° C.) for a duration in the range of about 1 hour to about 24 hours (such as 3 hours); and
iii) providing a paper-based sensor as described herein;
iv) adhering the cured first silicone layer of step (ii) to a first surface of the paper-based sensor of step (iii);
v) adhering a blood filtration membrane to a second surface of the paper-based sensor of step (iii), the second surface being opposite to the first surface; and
vi) adhering the cured second silicone layer to blood filtration membrane.

The first and second layers may be fabricated from medical grade silicone or medical grade acrylate (such as polymethyl methacrylate).

The first silicone layer may be regarded as the top silicone layer and is adhered to the top surface of the paper-based sensor which then forms a layer over the detection zones, while the second silicone layer may be regarded as the bottom silicone layer and is adhered to the bottom surface of the paper-based sensor with the blood filtration membrane in-between. The blood filtration membrane is thus adhered to the base of the paper-based sensor and the bottom silicone layer is adhered to the blood filtration membrane. The first silicone layer is thus synonymous with the top silicone layer and the second silicone layer is thus synonymous with the bottom silicone layer.

The silicone layer may be fabricated from a base elastomer component and a curing component, wherein the base elastomer may be poly(dimethylsiloxane). The base elastomer and the curing component may be mixed in a ratio of about 0.3:1 to about 30:1, about 0.6:1 to about 30:1, about 0.9:1 to about 30:1, about 10:1 to about 30:1, about 20:1 to about 30:1, about 0.3:1 to about 20:1, about 0.3:1 to about 10:1, about 0.2:1 to about 0.9:1 or about 03:1 to about 0.6:1.

The top and bottom silicone layers may be fabricated from medical grade silicone selected from the group consisting of SILASTIC@MDX4-4210 BioMedical Grade Elastomer, Liveo™ MG 7-9800 Soft Skin Adhesive, Liveo™ MG7-9900 Soft Skin Adhesive and Ecoflex™ silicone.

The top and bottom silicone layers may be commercial wound contact layers selected from the group consisting of 3M™ Tegaderm™ Transparent Film, OPSITE Flexiflex™, Auxano Suprathel® film and Mepitel Silicone Wound Contact Layer. The top and bottom silicone layers may be transparent.

The blood filtration membrane may be cellulose or glass microfiber. The blood filtration membrane may have a thickness of about 200 μm to about 400 μm, about 250 μm to about 400 μm, about 300 μm to about 400 μm, about 350 μm to about 400 μm, about 200 μm to about 350 μm, about 200 μm to about 300 μm or about 200 μm to about 250 μm.

The blood filtration membrane may have a capillary flow rate (wicking rate) of about 20 s/4 cm to about 50 s/4 cm, about 30 s/4 cm to about 50 s/4 cm, about 40 s/4 cm to about 50 s/4 cm, about 20 s/4 cm to about 40 s/4 cm or about 20 s/4 cm to about 30 s/4 cm. The capillary flow rate may be 29.7 s/4 cm or 35.6 s/4 cm. The blood filtration membrane may further have a water absorption of about 20 mg/cm² to about 100 mg/cm², about 40 mg/cm² to about 100 mg/cm², about 60 mg/cm² to about 100 mg/cm², about 80 mg/cm² to about 100 mg/cm², about 20 mg/cm² to about 80 mg/cm², about 20 mg/cm² to about 60 mg/cm² or about 20 mg/cm² to about 40 mg/cm². The water absorption may be 39.4 mg/cm² or 25.3 mg/cm². The blood filtration membrane may be Whatman MF1 or Whatman LF1.

Advantageously, the wound sensor patch produced by the method as described herein allows for extended shelf-life and for modular integration with other wound dressings.

Exemplary, non-limiting uses of the paper-based sensor as described herein will now be disclosed.

The paper-based sensor of the present disclosure may be used for determining wound biomarkers. The paper-based sensor of the present disclosure may be used for simultaneously determining wound biomarkers. The paper-based sensor of the present disclosure may be used for analysing wound biomarkers.

The paper-based sensor of the present disclosure may be applied directly to a wound for wound diagnosis and monitoring.

The paper-based sensor may be integrated in combination with other wound dressings to be applied to a wound for wound diagnosis and monitoring. Other wound dressing may be bandages, gauzes, adhesive wound dressings, transparent films, collagen dermal templates, hydrogel-like dressing materials or any combinations thereof.

Exemplary, non-limiting embodiments of a kit comprising the paper-based sensor, a device for capturing images and a software for image analysis will now be disclosed.

The paper-based sensor of the present disclosure may be integrated into a kit comprising a device for capturing images and a software for image processing.

The device for capturing images may be a mobile device, a digital camera or a mobile phone with integrated camera.

The captured image may then be analysed using an image processing software. The image processing software may be GIMP or ImageJ. The image processing software may be integrated into the device for capturing images, or may be on an external device, wherein the captured image may be transferred to the external device for image processing and analysis. The image may be transferred wirelessly.

### BRIEF DESCRIPTION OF THE DRAWINGS

The accompanying drawings illustrate disclosed embodiments and serve to explain the principles of the present disclosure. It is to be understood, however, that the drawings are designed for purposes of illustration only, and not as a definition of limits of the invention.

FIG. 1A shows a photograph of the paper-based sensor with five detection zones and their respective sensor positions (1) temperature detection zone, (2) trimethylamine detection zone, (3) pH detection zone, (4) moisture detection zone and (5) uric acid detection zone; FIG. 1B is a schematic diagram of the front and back view of the paper-based sensor with the diameter and widths of the detection zone and channels; and FIG. 1C is a cross-sectional schematic diagram of the channels, detections zone and sample zone

FIG. 2 shows a chart comprising photograph images of the optimization of wax heating time to create channels of suitable depth to achieve un-impeded flow of simulated wound fluid.

FIG. 3 shows a colourimetric chart comprising photograph images for the different cholesteric liquid crystals at different compositions at different temperatures. The CLCs are red at 31° C., green at 32° C., turquoise at 33° C., blue at 34° C., dark blue at 35° C. and very dark blue at 36° C.

FIG. 4 shows a chart comprising photograph images of the colour change of the cholesteric liquid crystals on cellulose paper substrate at various temperatures; red at 31° C., green at 32° C. and blue at 33° C.

FIG. 5 shows a photograph of the response of Reichardt's dye on non-treated paper and paper treated with perfluorooctyl trimethoxy silane. The response is dark grey at 0 ppm to 50 ppm, grey at 100 ppm to 300 ppm and bright grey at more than 500 ppm of trimethylamine concentration.

FIG. 6A shows a chart comprising photograph images of the colourimetric response of Reichardt's dye with various concentrations of trimethylamine; FIG. 6B is a graph showing change in luminance values at various trimethylamine concentrations using GIMP Image analysis; and FIG. 6C is a graph showing change in luminance values at relevant wound monitoring range at 30-300 ppm of trimethylamine.

FIG. 7 shows a photograph of the response of the pH sensor of various layers to pH 8.4 buffer solution.

FIG. 8 shows both the calibration curve of the pH sensor and photograph images of the pH sensor in response to different known pH values. The response is approximately dark yellow from pH 2.5 to pH 4, yellow from pH 4 to pH 6.5, light yellow from pH 6.5 to pH 7.5, orange from pH 7.5 to pH 8, pink from pH 8 to pH 9.5 and magenta from pH 9.5 and more.

FIG. 9 shows both the calibration curve of the moisture sensor and photograph images of the moisture sensor at different moisture percentage levels. The response is approximately blue from moisture levels 0% to 30%, violet from moisture levels 30% to 60% and pink from moisture levels 60% to 100%.

FIG. 10 show a chart comprising photograph images of tuning the chitosan matrix at different pH levels (pH 5.5, pH 6.5 and pH 7.5).

FIG. 11 shows both a graph of Gray value against uric acid concentration to compare the enzymatic activity for enzymes dissolved in phosphate buffered saline and stabilizer solution, and a chart comprising of photograph images of the colourimetric response at various uric acid concentrations for enzymes dissolved in phosphate buffered saline and stabilizer solution

FIG. 12 shows a chart comprising photograph images of the colour gradient of the uric acid sensor loaded on different Whatman filter paper (Grades 1, 2 and 3) at various uric acid concentrations.

FIG. 13 shows a chart comprising photograph images comparing the false positive signal for 3,3′,5,5′-tetramethybenzidine and 4-aminoantipyrine at various uric acid concentration at 5 minutes and at 1 hour.

FIG. 14 shows both a calibration curve of uric acid concentration and chart comprising photograph images of the uric acid sensors at various uric acid concentrations. The response is approximately colourless at uric acid concentrations from 0 μM to 40 μM, light pink from 40 μM to 100 μM, pink from 100 UM to 400 μM and deep pink from 400 μM and more.

FIG. 15 shows the colourimetric response comprising of photograph images of the paper-based sensor with samples in phosphate buffered saline of various concentrations added directly to the individual detection zones.

FIG. 16 shows a chart comprising of photograph images of the colourimetric response of the paper-based sensor at two simulated wound exudates (healthy and unhealthy) taken at time t=0 minutes, t=1 minutes, t=3 minutes, t=8 minutes and t=15 minutes.

FIG. 17A shows the colourimetric response of the paper-based sensor “before addition” of simulated wound exudates added to the sample zone from the back of the sensor; FIG. 17B shows the colourimetric response of the paper-based sensor in a “healthy state” of simulated wound exudates added to the sample zone from the back of the sensor; FIG. 17C shows the colourimetric response of the paper-based sensor in an “unhealthy state” of simulated wound exudates added to the sample zone from the back of the sensor; FIG. 17D is a quantitative chart of the result in the temperature detection zone; FIG. 17E is a quantitative chart of the result in the TMA detection zone; FIG. 17F is a quantitative chart of the result in the pH detection zone; FIG. 17G is a quantitative chart of the result in the moisture detection zone; and FIG. 17H is a quantitative chart of the result in the uric acid detection zone.

FIG. 18 shows a diagram of a wound sensor patch comprising the paper-based sensor between a top and bottom silicone layer, wherein between the bottom silicone layer and the bottom of the paper-based sensor is a circular blood filtration membrane.

### EXAMPLES

Non-limiting examples of the invention will be further described in greater detail by reference to specific examples, which should not be construed as in any way liming the scope of the invention.

### Example 1: Wax Protecting the Paper Base with Fluidic Pattern

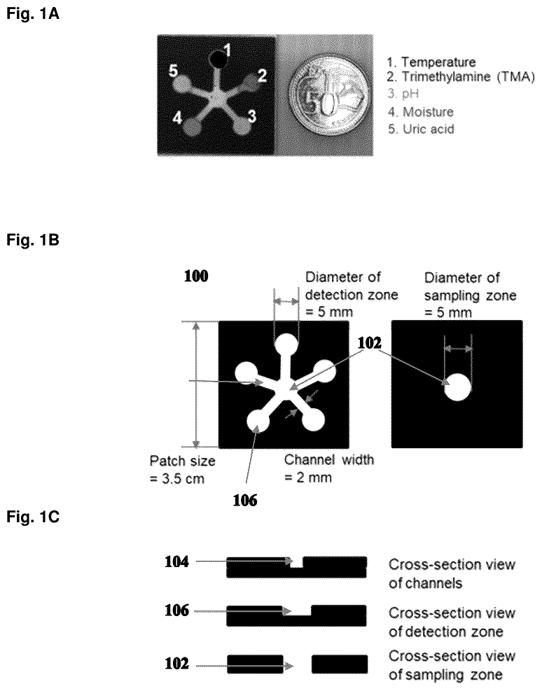

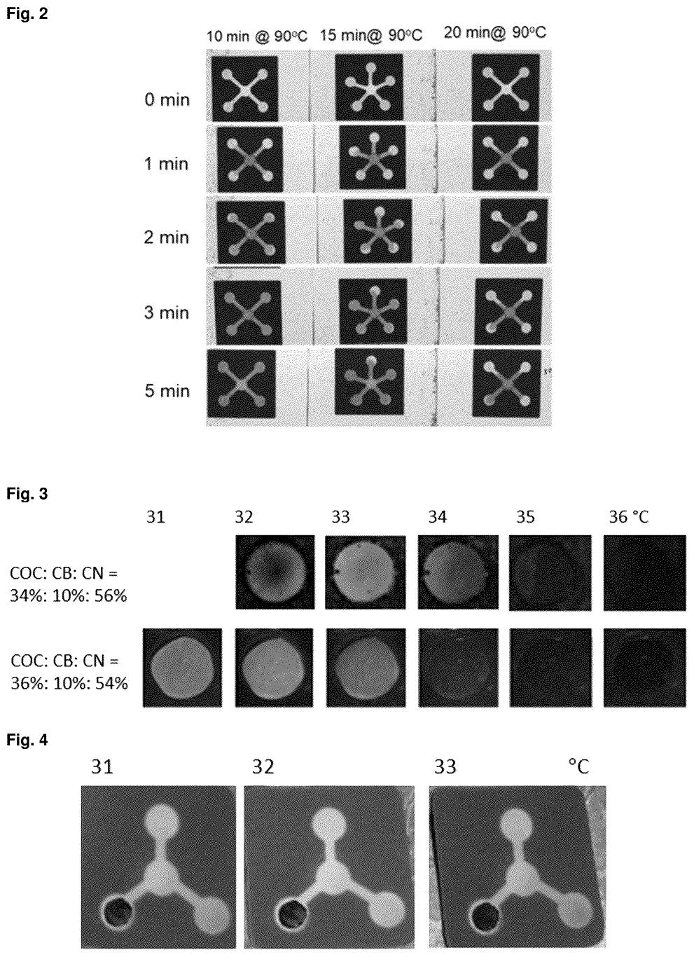

Whatman filter paper Grade 3 with particle retention of 6 μm and thickness of 390 μm (purchased from Sigma Aldrich, St. Louis, Untied States) was selected to be the base for the paper-based sensor. Wax (ink cartridge purchased from Xerox, Norwalk, Connecticut, United States) was printed with a fluidic pattern on the base substrate leaving only the detection zones, channels and sample zone as seen from FIG. 1. The pattern was prefabricated using computer-aided design (CAD) to mark the boundaries of the detection zones, channels and sample zones. Subsequently, the wax was printed on the paper based on the CAD pattern. The wax was then heated at 90° C. for 10 minutes to melt the wax for penetration into the base substrate and then cooled in air, therefore wax protecting the base substrate, leaving only the detection zones, channels and sample zone, thus forming the paper-based sensor. Therefore, as shown in FIG. 1, there is provided a paper-based sensor 100 comprising a sample zone 102 connected via channels 104 to a plurality of detection zones 106 where the detection zones 106 are considered to be in fluid communication with the sample zone 102. As seen from FIG. 2, the optimized heating was found at 90° C. for 10 minutes to melt the wax for penetration.

### Example 2: Preparation and Optimization of the Temperature Detection Zone

The temperature detection zone of the paper-based sensor was prepared using cholesteric liquid crystals (CLCs) solution comprising three components of cholesterol derivatives; cholesteryl oleyl carbonate (COC); cholesteryl benzoate (CB); and cholesteryl nonanoate (CN) (obtained from Sigma Aldrich, St. Louis, Missouri, United States). The CLC mixture was first prepared by melting the CLCs at 100° C. for 1 hour. As seen from FIG. 3, the ratio of the three cholesterol derivatives was optimized to be 36% COC, 10% CB and 54% CN. As seen from FIG. 4, the temperature detection zone on the paper-based sensor was treated with black ink (from a permanent marker) and then dried at room temperature for 15 minutes, onto which, 2 mg of the melted CLC mixture was then pasted and spread on top.

### Example 3: Preparation and Optimization of the Trimethylamine Detection Zone

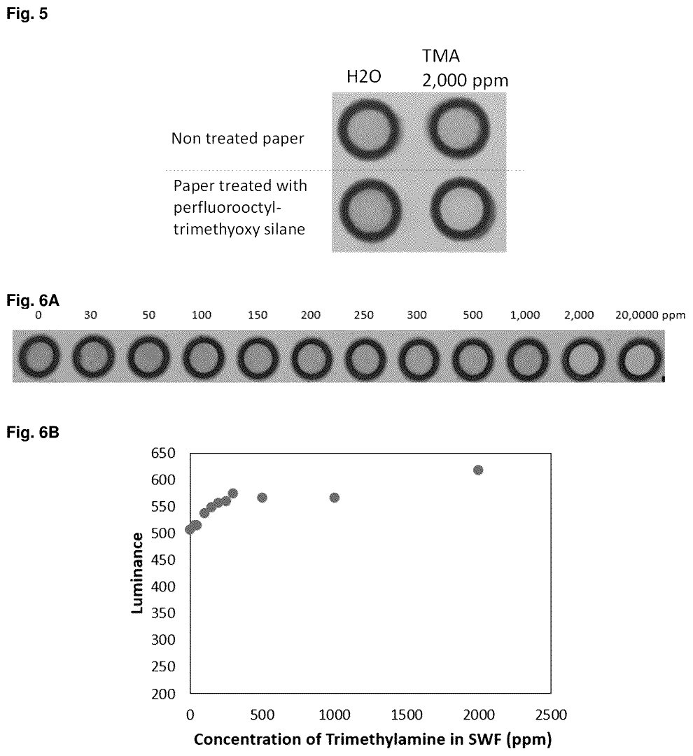

The TMA detection zone of the paper-based sensor was prepared from Reichardt's dye purchased from Sigma Aldrich (St. Louis, Missouri, United States). A solution of 5 mg/ml of Reichardt's dye in ethanol was first prepared. The TMA detection zone on the paper-based sensor was then treated with 2 μL of 1% perfluorooctyl-trimethoxy silane (purchased from Sigma Aldrich, St. Louis, Missouri, United States) and dried at room temperature for 10 minutes. 2 μL of the as prepared 5 mg/mL Reichardt's dye in ethanol was then drop-casted onto the TMA detection zone treated with 1% perfluorooctyl-trimethoxy silane and dried at 40° C. for 10 minutes. As seen from FIG. 5, the response on the 1% perfluorooctyl-trimethoxy silane treated detection zone shows improved colour response.

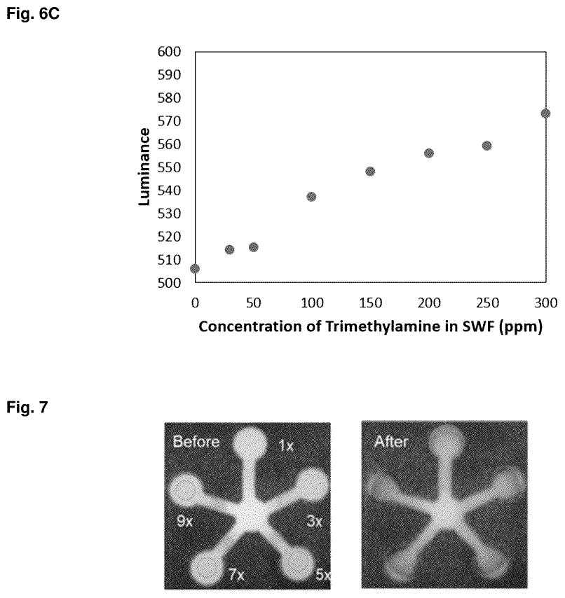

The prepared TMA detection zone was then calibrated using various TMA concentrations from 0 ppm to 20 000 ppm. The various concentrations of TMA were applied to the prepared TMA detection zone and left for 30 minutes to allow full reaction and colour change. Digital photographs of the colour change were taken and analysed using GIMP software to obtain the luminance value. The change in luminance was used to correlate the colour change with TMA concentration. The optimized TMA detection zone was found to have a dynamic range between 0 ppm to 3 000 ppm as seen from FIG. 6B. In particular, the optimized TMA detection zone has relevant wound monitoring range between 30 ppm to 300 ppm as seen from FIG. 6C.

### Example 4: Preparation and Optimization of the pH Detection Zone

The pH detection zone of the paper-based sensor was prepared from 0.04% of phenol red powder purchased from Sigma Aldrich (St. Louis, Missouri, United States) dissolved in water. 2 μL of the phenol red solution was then drop-casted onto the pH detection zone on the paper base and dried at room temperature for 5 minutes to produce one layer of pH detection zone. A second 2 μL of the phenol red solution was then drop-casted onto the first dried layer and dried at room temperature for 5 minutes to produce two dried layers of the pH detection zone. A third and final 2 μL of the phenol red solution was then drop-casted onto the second dried layer and dried at room temperature for 5 minutes to produce three dried layers of the pH detection zone.

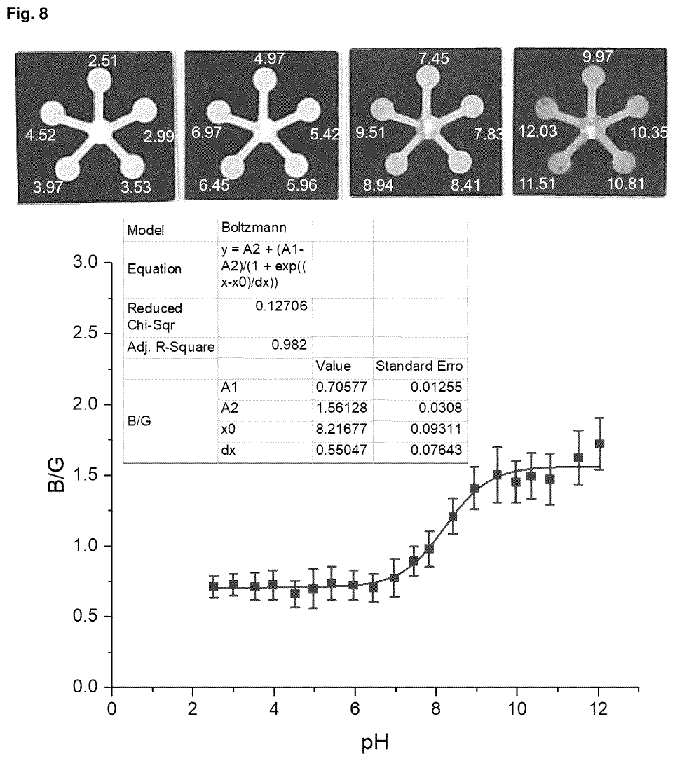

The prepared pH detection zone was then calibrated using 3 μL of buffers (self-prepared) with pH from about pH 2 to about pH 12. The buffers were left for 5 minutes on the pH sensors to allow for a full development of the colour change to occur, as seen from FIG. 8. Digital photographs of the colour change were taken and analysed using ImageJ software to obtain the Red-Blue-Green (RGB) values and the ratio of B/G values was used to correlate the colour change with the pH values. The optimized pH detection zone of the paper-based sensor was found to have a dynamic range between pH 6-10 with a resolution of approximately 0.5 pH as seen from FIG. 9.

### Example 5: Preparation and Optimization of the Moisture Detection Zone

The moisture detection zone of the paper-based sensor was prepared from 100 mg/ml of anhydrous cobalt chloride (CoCl₂) dissolved in 2 weight % (20 mg/mL) polyhydroxyethylmethacrylate (pHEMA)/ethanol (both CoCl₂ and pHEMA were purchased from Sigma Aldrich, St. Louis, Missouri, Untied States). Depending on the size of the detection zone, 10 L/cm² of (the zone size can be varied to tailor to the final wound size, therefore the volume of reagent will also vary depending on the zone size.) of the CoCl₂ solution was drop-casted onto the moisture detection zone on the paper base and dried at 25° C. in an over at 100% fan speed for 5 minutes to produce a first layer. A second 10 L/cm² of the CoCl₂ solution was drop-casted onto the first dried layer and dried at 25° C. in an oven at 100% fan speed for 5 minutes to produce a second dried layer of moisture detection zone. A third and final 10 L/cm² was drop-casted onto the second dried layer and dried at 25° C. in an oven at 100% fan speed for 5 minutes to produce the third dried layer of the moisture detection zone.

The prepared moisture detection zone was then calibrated at different moisture level ranging from about 0% to about 100% moisture for about 5 minutes to allow for full colour development. The different moisture levels were achieved through mixing various ratios of water and ethanol (for example, 50% moisture level was prepare using 1:1 water to ethanol mixture). Digital photographs of the colour change were taken and analysed using ImageJ software to obtain the RGB values and the ratio of R/B values was used to correlate colour change with moisture. The optimized moisture detection zone of the paper-based sensor was found to have dynamic range between 0% to 40% moisture with a limit of detection of 10% moisture as seen from FIG. 9.

### Example 6: Preparation and Optimization of the Uric Acid detection zone

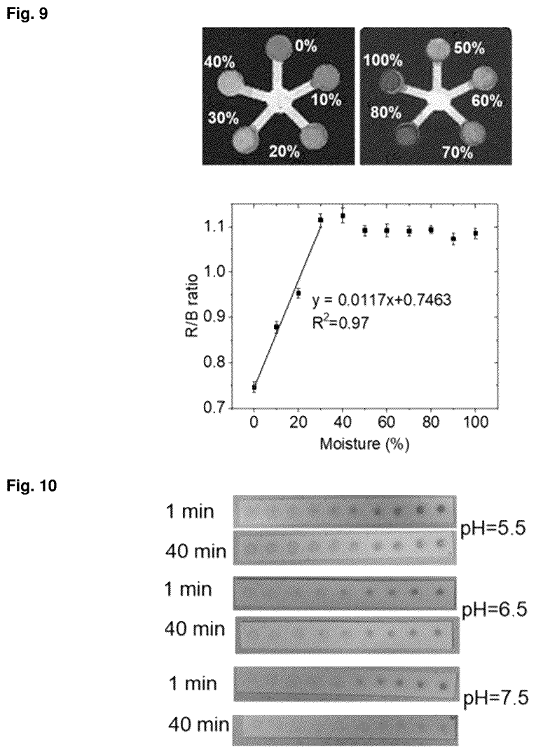

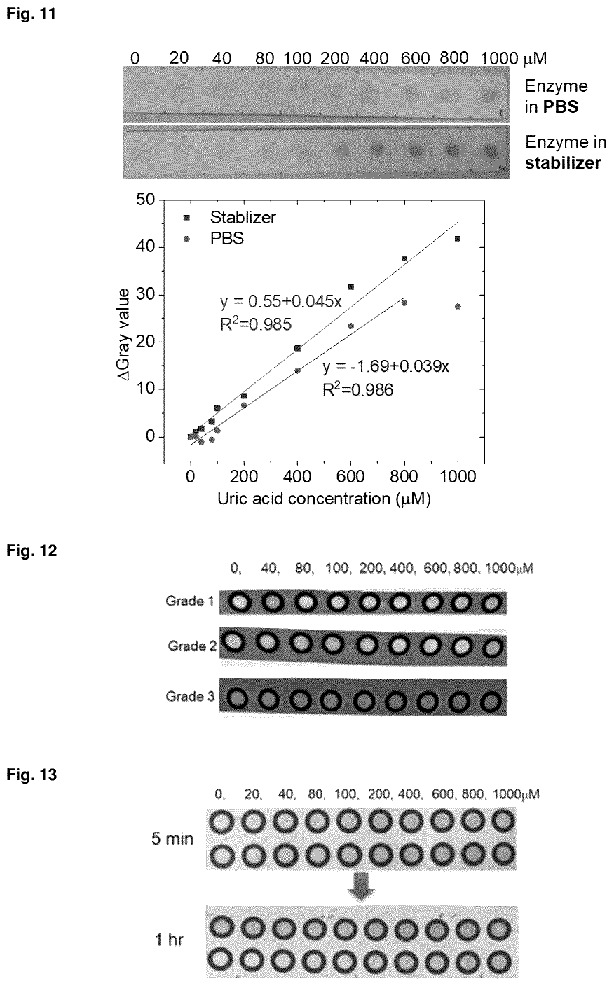

The uric acid detection zone of the paper-based sensor was prepared from stabilized uricase enzymes with 4-aminoantipyrine (AAP) enzymatic substrate. 1 μL of 1 weight % chitosan (obtained from Sigma Aldrich, St. Louis, Missouri, Untied States) at pH 6.5 was drop-casted onto the uric acid detection zone on the cellulose paper base and dried at room temperature for 5 minutes to produce the uric acid sensor matrix. Next, 1 L of 16 mM AAP (obtained from Sigma Aldrich, St. Louis, Missouri, Untied States) dissolved in 8 mM sodium 3,5-dichloro-2-hydroxybenzenesulfonate (DHBS) (obtained from Sigma Aldrich, St. Louis, Missouri, Untied States) was drop-casted onto the same detection zone and dried at room temperature for 5 mins. Next, 1 μL of 0.15 mg/mL horseradish peroxidase (HRP) (obtained from Sigma Aldrich, St. Louis, Missouri, Untied States) in StabilCoat@ Immunoassay Stabilizer solution (obtained from Sigma Aldrich, St. Louis, Missouri, Untied States) was drop-casted onto the same detection zone and dried at room temperature for 5 minutes. Next, 1 μL of 40 mg/mL uricase in StabilCoat® Immunoassay Stabilizer solution was drop-casted onto the same detection zone and dried at room temperature for 5 minutes. Lastly, 1 μL of 16 mM AAP dissolved in 8 mM DHBS was drop-casted again onto the same detection zone and dried at room temperature for 5 minutes. As seen from FIG. 10, a good colour change is observed when the uric acid sensor is fabricated with a chitosan matrix at pH 6.5. As seen from FIG. 11, the colour change is well maintained for stabilized uric acid sensor. As seen from FIG. 12, a good colour gradient is obtained on Whatman filter paper Grade 3. As seen from FIG. 13, the use of AAP prevents false positive signals.

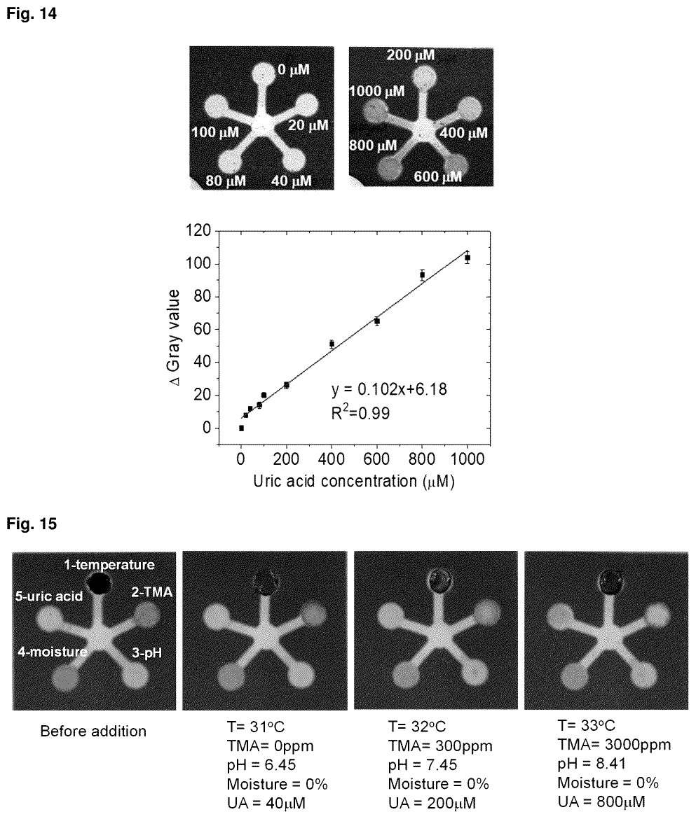

The prepared uric acid detection zone was then calibrated using various concentrations of uric acid from 0 μM to 1 000 μM. Digital photographs of the uric acid sensor with no uric acid (0 μM) were taken under room light conditions to establish the background colour. Various concentrations of uric acid were then added to sensor and the colour change allowed to stabilize for 10 minutes. Subsequently, digital photographs of the colour change were taken and analysed using ImageJ software to obtain the intensity values of the detection zones. The intensity values were then background subtracted and plotted against uric acid concentrations to construct a calibration curve. The optimized uric acid detection zone was found to have a dynamic range between 40 μM to 1 000 μM with a limit of detection of 40 μM uric acid concentration, as seen from FIG. 14.

### Example 7: Demonstration of Biomarker Sensors in Paper-Based Sensor

A paper-based sensor was prepared comprising a wax pattern of five detection zones coupled with five channels coupled to a sample zone printed and sealed onto Whatman Grade 3 filter paper. The paper-based sensor was initially tested for responsiveness and change in colour using target analytes dissolved in phosphate buffer saline (PBS) which was then individually applied directly to each of the detection zones containing the sensor. As seen from FIG. 15, as temperature increased from 31° C. to 32 to 33° C., the temperature detection zone changed from Red/Orange to Green to Dark Blue; as TMA concentrations increased from 0 ppm to 300 ppm to 3 000 ppm, the TMA detection zone changed from grey to light grey to off-white; as pH increased from pH 6.45 to pH 7.45 to pH 8.41, the pH detection zone changed from Yellow to Light Orange and to Dark Orange; and as uric acid concentration increased from 40 μM to 200 μM to 800 μM, the uric acid detection zone changed from Light Pink to Pink to an intense Pink. Due to the reversibility of the moisture detection zone, it remained blue as the sensor was dried on a hotplate after application of the target analytes (moisture is recorded at 0%).

Referring to FIG. 15, the responses for the detection zones are as follows:

TABLE 2
Summary of colour change responses of FIG. 15

Location /

| Biomarker | Condition and Colour Responses | | |
|---|---|---|---|
| 1 - Temperature | 31° C. - dark red/green | 32° C. - green | 33° C. - blue |
| 2 - TMA | 0 ppm - dark grey | 300 ppm - grey | 3 000 ppm - bright grey |
| 3 - pH | pH 6.45 - yellow | pH 7.45 - orange | pH 8.41 - light pink |
| 4 - Moisture | 0% after drying - blue | 0% after drying - blue | 0% after drying - blue |
| 5 - Uric acid | 40 μM - colourless | 200 μM - light pink | 800 μM - pink |

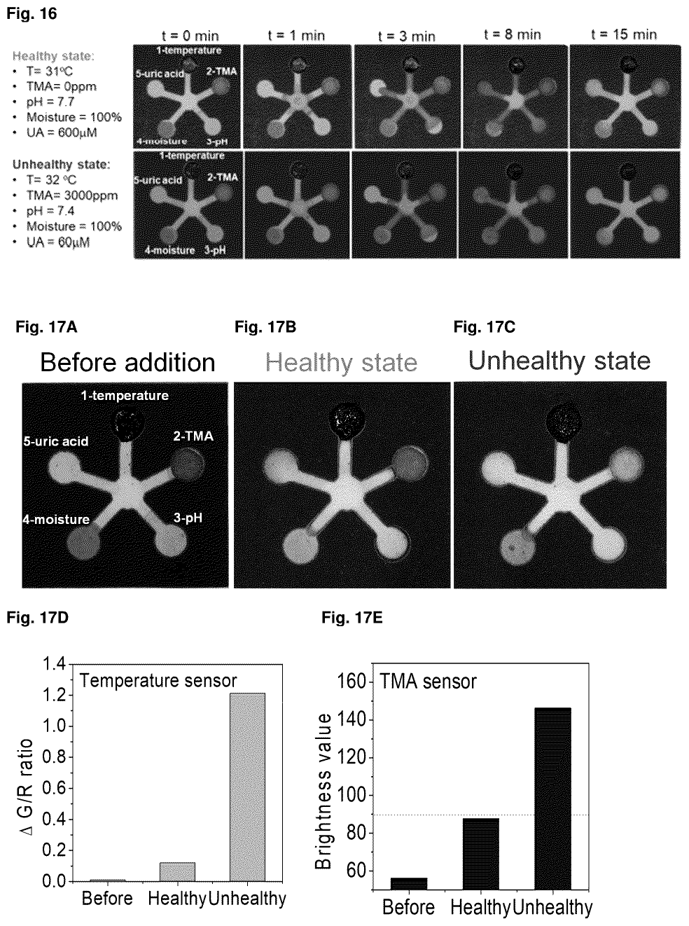

To demonstrate the paper-based sensor under wound exudate flow conditions, a simulated health and unhealthy wound exudate fluids were prepared and applied to the back of the sample zones of two paper-based sensors. Digital photographs were taken at time t=0 minute, t=1 minute, t=3 minutes, t=8 minutes and t=15 minutes. As seen from FIG. 16, the paper-based sensors were fully wet after 8 minutes, showing no further colour change of the sensors in the detection zones.

Referring to FIG. 16, the responses for the detection zones are as follows:

TABLE 3
Summary of colour change responses of FIG. 16

| Location / | | | | | |
|---|---|---|---|---|---|
| Biomarker / | Time | Time | Time | Time | Time |
| Simulated wound | t = 0 min | t = 1 min | t = 3 mins | t = 8 mins | t = 15 mins |
| 1 - Temperature | | | | | |
| Healthy - 31° C. | green | green | green/red | dark red | dark red |
| Unhealthy - 32° C. | dark green | green | red | dark red | dark red |
| 2 - TMA | | | | | |
| Healthy - 0 ppm | grey | grey | grey | grey | grey |
| Unhealthy - 3 000 | grey | grey | grey | grey | bright grey |
| ppm | | | | | |
| 3 - pH | | | | | |
| Healthy - 7.7 pH | yellow | yellow | dark yellow | orange | orange |
| Unhealthy - 7.4 pH | yellow | yellow | yellow | yellow | yellow |
| 4 - Moisture | | | | | |
| Healthy - 100% | blue | blue | violet | pink | pink |
| Unhealthy - 100% | blue | blue | violet | pink | pink |
| 5 - Uric acid | | | | | |
| Healthy - 600 μM | colourless | colourless | colourless | dark blue | pink |
| Unhealthy - 60 μM | colourless | colourless | colourless | dark blue | colourless |

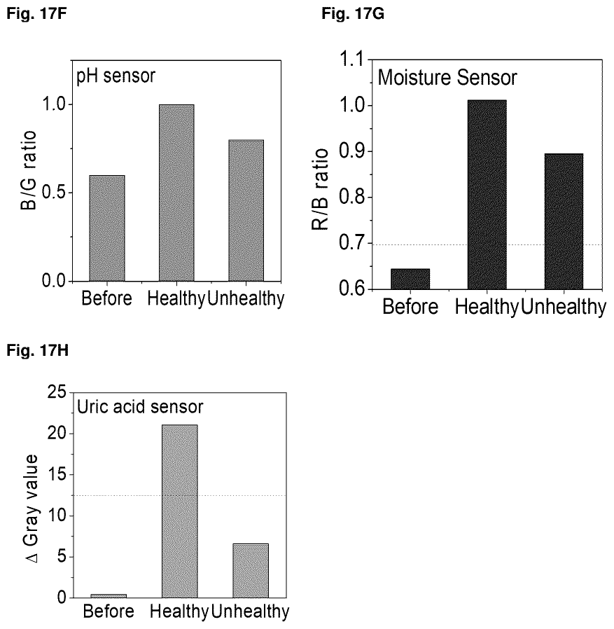

By applying the simulated wound exudate from the back of the sample zone, it simulated how the paper-based sensor would be applied to a wound for wound monitoring and diagnosis. The prominent colour changes were observed not only visually (see FIGS. 17A, 17B and 17C) but quantitative colour analysis using ImageJ software on the digital photographs revealed distinctive signal difference between the simulated healthy and unhealthy wound exudate as seen from FIG. 17D to 17H.

Referring to FIG. 17, the responses for the detection zones are as follows:

TABLE 4
Summary of colour change responses of FIG. 17

| Location / | Before | Healthy healing | Unhealthy non- |
|---|---|---|---|
| Biomarker | addition | wound state | healing wound state |
| 1 - Temperature | dark red | dark red | dark green |
| 2 - TMA | dark grey | grey | bright grey |
| 3 - pH | yellow | orange | yellow |
| 4 - Moisture | blue | pink | blue |
| 5 - Uric acid | colourless | pink | very light pink |

### Example 8-Preparing a Wound Sensor Patch using the Paper-based Sensor

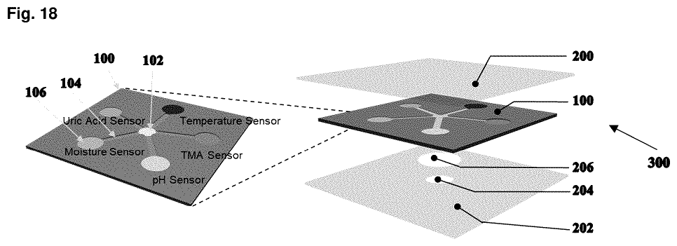

As seen from FIG. 18, the paper-based sensor can be incorporated between a top and bottom silicone layer, with a circular blood filtration membrane between the bottom surface of the paper-based sensor and the bottom silicone layer. The top silicone layer was fabricated from medical grade silicone MDX4-4210 (obtained from Dupont, Delaware, United States) by mixing 500 mg base agent with 50 mg curing agent and curing at 60° C. for 3 hours. The bottom silicone layer was fabricated from medical grade MG7-9800 (obtained from Dupont, Delaware, United States) by mixing 1.35 mL of Part A of the MG7-9800 kit with 1.5 mL of Part B of the MG7-9800 kit and cured at 60° C. for 3 hours. The blood filtration membrane was Whatman MF1 (obtained from Maidstone, United Kingdom) and was cut to a circular form matching the diameter of the sample zone. Upon forming the top and bottom transparent silicon layers, the top silicone layer was adhered to the top side of the paper-based sensor and the bottom silicone layer was adhered to the bottom side of the paper-based sensor with the circular blood filtration membrane aligned with the sample zone on the bottom side. The four layers constituted the wound sensor patch for modular integration with other wound dressings.

As seen from FIG. 18, there is provided a wound sensor patch 300 comprising the paper-based sensor 100 with a top transparent silicone layer 200 and a bottom transparent silicone layer 202 having a circular opening 204, and a blood filtration membrane 206 in between the bottom surface of the paper-based sensor 100 and the bottom silicone layer 202. The paper-based sensor 100 comprises a sample zone 102 connected via channels 104 to a plurality of detection zones 106 where the detection zones 106 are considered to be in fluid communication with the sample zone 102.

### Summary of Examples

The wax protected cellulose paper with one or more printed detection zone, channels and sample zone, in combination with one or more of the sensing materials as described herein, enables the production of a paper-based sensor capable of monitoring wound health within minutes without causing additional trauma to the wound. A summary of the biomarkers (denoting the detection zones) and their sensing materials is presented in Table 5. Below.

### INDUSTRIAL APPLICABILITY

In the present disclosure, the paper-based sensor as described herein may be used for wound monitoring. The sensor may be manufactured into a wound sensor patch to provide a real-time assessment of wound healing status through five spectrometric sensors (denoted as detection zones) which provide quantitative characterizations of temperature, trimethylamine, pH, moisture and uric acid. Further, the paper-based sensor as described herein may provide a way to monitor wounds without removal of wound dressing with quicker assessments of wound infection within minutes due to the quick response of the colourimetric sensors.

It will be apparent that various other modifications and adaptations of the invention will be apparent to the person skilled in the art after reading this foregoing disclosure without departing from the spirit and scope of the invention and it is intended that all such modifications and adaptations come within the scope of the appended claims.

1. A paper-based sensor for simultaneously determining a plurality of biomarkers present in a biological sample, said paper-based sensor comprising:
a) a sample zone for receiving said biological sample containing said plurality of biomarkers; and
b) a plurality of detection zones in fluid communication with said sample zone, each of said plurality of detection zones comprising sensing material specific to each of said plurality of biomarkers,
wherein said plurality of biomarkers are selected from the group consisting of temperature, trimethylamine (TMA), pH, moisture, and uric acid.

2. The paper-based sensor of claim 1, wherein each of said detection zones is connected to said sample zone via respective channels.

3. The paper-based sensor of claim 1, further comprising a base selected from a cellulose base, nitrocellulose base, and glass microfibers.

4. The paper-based-sensor of claim 1, wherein each of said detection zones extends radially outwards from said sample zone.

5. The paper-based sensor of claim 1, further comprising a hydrophobic region surrounding said sample zone and said detection zones, wherein said hydrophobic region comprises wax.

6. The paper-based sensor of claim 1, wherein when the biomarker is temperature, the sensing material for said temperature biomarker comprises a mixture of cholesteric liquid crystals (CLCs).

7. The paper-based sensor of claim 1, wherein when the biomarker is trimethylamine, and the sensing material for said trimethylamine biomarker comprises a solvatochromic dye dissolved in alcoholic solvent.

8. The paper-based sensor of claim 1, wherein when the biomarker is pH, and the sensing material for said pH biomarker comprises aqueous phenol red, neutral blue, or bromothymol blue.

9. The paper-based sensor of claim 1, wherein when the biomarker is moisture, and the sensing material for said moisture biomarker comprises a transition metal salt dissolved in a mixture of polyhydroxyethylmethacrylate/alcoholic solution.

10. The paper-based sensor of claim 1, wherein when the biomarker is uric acid, the sensing material for said uric acid biomarker comprises uricase enzyme in a stabiliser stabilizer solution in an enzymatic substrate in sodium 3,5-dichloro-2-hydroxybenzenesulfonate (DHBS) with horseradish peroxidase (HRP) in stabilizer solution on a biopolymer matrix.

11. A method of manufacturing the paper-based sensor of claim 1, comprising the steps of:
a) providing a pattern having a first area and a second area, wherein the first area defines a sample zone and a plurality of detection zones in fluid communication with the sample zone and wherein the second area is a remaining portion of the base substrate that is not covered by the first area;
b) printing a hydrophobic material or ink onto the second area of the pattern of step (a);
c) heating the hydrophobic material or ink to demarcate the sample zone and the plurality of detection zones in the first area; and
d) treating each of said plurality of detection zones with a sensing material that is specific to a biomarker selected from the group consisting of temperature, TMA, pH, moisture, and uric acid.

12. The method according to claim 11, wherein the treating step for the sensing material for said temperature biomarker comprises the steps of:
a) melting a cholesteric liquid crystal (CLC) mixture at a temperature of 80° C. to 120° C. for 30 minutes to 2 hours;
b) depositing a black ink on the temperature detection zone and drying for 5 minutes to 20 minutes at a temperature of 25° C. to 50° C.; and
c) dropping and spreading an amount of the melted CLC of step (a) onto the temperature detection zone of step (b).

13. The method according to claim 11, wherein the treating step for the sensing material of said TMA biomarker comprises the steps of:
i) dissolving 1 mg/mL to 10 mg/ml of a solvatochromic dye selected from the group consisting of Reichardt's dye, Nile Red, and 3-hydroxychromonesin in an alcohol;
ii) treating the TMA detection zone with 1% to 10% perfluorooctyl-trimethoxy silane;
iii) drop casting 10 μL/cm² to 30 μL/cm² of the solution prepared in step (i) into the treated TMA detection zone of step (ii); and
iv) drying the TMA detection zone for 5 minutes to 20 minutes at a temperature of about 25° C. to about 50° C. after step (iii).

14. The method according to any claim 11, wherein the treating step for the sensing material of said pH biomarker comprises the step of:
i) dissolving 0.02% to 0.10% of phenol red, neutral red, or bromothymol blue in water to form a solution;
ii) drop casting 10 μL/cm² to 30 μL/cm² of the solution prepared from step (i) into the pH detection zone;
iii) air drying the pH detection zone in step (ii) for 5 minutes to 20 minutes at a temperature of 25° C. to 50° C. to create one layer; and
iv) repeating steps (ii) and (iii) to produce up to 3 layers.

15. The method of claim 11, wherein the treating step for the sensing material for said moisture biomarker comprises the steps of:
i) dissolving 10 mg/mL to 200 mg/ml of a transition metal salt in 2 wt % (20 mg/mL) polyhydroxyethylmethacrylate (pHEMA)/ethanol solution;
ii) drop casting 10 μL/cm² to 30 μL/cm² of the solution prepared in step (i) into the moisture detection zone;
iii) drying the moisture detection zone in step (iii) in an oven at 20° C. to 30° C. for 5 minutes to 20 minutes to create one layer; and
iv) repeating steps (ii) and (iii) to produce up to 3 layers.

16. The method of claim 11, wherein the treating step for the sensing material for said uric acid biomarker comprises the steps of:
i) adding 10 μL/cm² to 30 μL/cm² of 1 wt % to 100 wt % of a biopolymer matrix with pH tuned in the range of pH 5.5 to pH 7.5 to the uric acid detection zone and drying for 5 minutes to 20 minutes at room temperature;
ii) adding 10 μL/cm² to 30 μL/cm² of 10 mM to 100 mM of 3,3′,5,5′-tetramethylbenzidine (TMB),4-aminoantipyrine (AAP), o-phenylenediamine (OPD), o-dianisidine, or 2,2′-Azino-Bis-3-Ethylbenzothiazoline-6-Sulfonic Acid (ABTS) in 2 mM to 20 mM of 3,5-dichloro-2-hydroybenzenesulfonate (DHBS) to the uric acid detection zone in step (i) and drying for 5 minutes to 20 minutes at a temperature of 25° C. to 30° C.;
iii) adding 10 μL/cm² to 30 μL/cm² of 0.1 mg/mL to 1 mg/ml of horseradish peroxidase (HRP) in stabilizer solution to the uric acid detection zone in step (ii) and drying for 5 minutes to 20 minutes at a temperature of 25° C. to 30° C.;
iv) adding 10 μL/cm² to 30 μL/cm² of 10 mg/mL to 100 mg/ml of uricase in stabilizer solution to the uric acid detection zone in step (iii) and drying for 5 minutes to 20 minutes at a temperature of 25° C. to 30° C.; and
v) re-adding of 10 μL/cm² to 30 μL/cm² of the AAP solution in step (ii) to the uric acid detection zone in step (iv) and drying for 5 minutes to 20 minutes at a temperature of 25° C. to 30° C.

17-18. (canceled)

19. A kit comprising the paper-based sensor of claim 1, a device for capturing images and a software for image analysis.

20. The kit according to claim 19, wherein the device for capturing images is configured to capture an optical image of the paper-based sensor according to claim 19 and wherein the captured image is to be analyzed by the software for image analysis.

21. A method of diagnosing wound health, comprising the step of applying the paper-based sensor of claim 1 onto a wound directly or in combination with other wound dressings.
